(19) United States
(12) Patent Application Publication
(10) Pub. No.: US 2005/0019905 A1
(43) Pub. Date: Jan. 27, 2005

(54) System for containing and processing small objects

(75) Inventors: Dale N. Larson, Waban, MA (US); Edward E. Harlow JR., Boston, MA (US)

Correspondence Address: CHARLES G. CALL, 68 HORSE POND ROAD, WEST YARMOUTH, MA, 02673-2516, US

(73) Assignee: The President and Fellows of Harvard College

(21) Appl. No.: 10/625,860

(22) Filed: Jul. 23, 2003

(51) Int. Cl. C12M 1/12

(52) U.S. Cl. 435/297.5; 435/306.1

(57) ABSTRACT

Methods and apparatus for intermixing small solid objects and a liquid employed in a platform useful for affinity purification of protein samples in a high throughput format. The system handles up to 96 samples at a time and provides thorough mixing of affinity media with a lysate solution containing the protein samples which accelerates interaction between the media and the sample. Sample processing times are shortened and in some cases non-specific contamination is reduced.

### CROSS REFERENCE TO RELATED APPLICATIONS

This application is a Non-Provisional of U.S. Patent Application Ser. No. 60/387,821 filed Jul. 23, 2002.

### FIELD OF THE INVENTION

This invention relates to methods and apparatus for containing and processing small objects and more particularly, although in its broader aspects not exclusively, methods and apparatus for performing protein purifications manually, semi-automated, and fully automated operations.

### BACKGROUND OF THE INVENTION

Understanding gene function has become a major focus of life science and biotech/pharmaceutical research, and understanding proteins is central to understanding gene function. Numerous techniques may be used to purify proteins for analysis. See, for example: Protein Purification Techniques: A Practical Approach by Simon Roe (Editor, 2nd edition (April 2001) Oxford University Press; ISBN: 0199636745; Protein Purification: Principles and Practice by Robert K. Scopes, 3rd edition (January 1994) Springer Verlag; ISBN: 0387940723; and Protein Analysis and Purification: Benchtop Techniques by Ian M. Rosenberg, 1st edition (September 1996) Springer Verlag; ISBN: 0817637176.

Life science and the study of proteins requires high throughput techniques. Immunoprecipitation and other affinity methods for protein activities require new tools to provide the needed high throughput. High throughput expression and purification of proteins is important in several areas of research, including in vitro study of protein-protein interactions, the study of protein complexes, high throughput screening of small molecule ligands against protein targets, creation of protein arrays, and high throughput structural genomics (protein structure via NMR and/or X-ray crystallography). These activities are performed in biotechnology and pharmaceutical companies, as well as in academic research.

### SUMMARY OF THE INVENTION

The present invention is a general purpose consumable/disposable plate or custom pipette tips that facilitate the handling of many small objects. The invention may be used to advantage with any available solid-phase purification protocol, latex bead-based protein purification being just one example. When used in combination with robotic handling, the invention is well-suited to high-throughput research applications

In a preferred embodiment of the invention, a plate is fabricated to define a plurality of wells or cavities having a permeable bottom or sidewalls for holding small objects such as affinity beads to which the proteins in a lysate become bound. During purification, the wells may be immersed in the lysate one or more times until the binding of the protein to the affinity beads is complete. A flow of wash solution is then passed through the beads and through the permeable walls of the wells to wash the beads. A flow of elution solution may then be passed through the beads and the well walls to complete the purification process before the purified sample is collected. The well may be constructed of any inert material which will not affect the analysis being performed. A substantial portion of the bottom and/or lower sidewall of the web should be permeable to the liquids brought into contact with the beads or other material used to perform the analysis, but should retain the beans or other material during the analysis.

The invention provides a dramatic reduction in labor when purification is performed manually and further enables automated, unattended purification to be performed with high throughput. The technique is also applicable to situations where a number of small objects need to be contained and exposed to different fluids. Examples include protein purification where beads are used, and larvae and embryo staining. The technique is compatible with existing infrastructures for both manual and robotics applications. The consumable and disposable plates contemplated by the invention may be used to advantage in the high-throughput purification of proteins and for immunoprecipitation.

These and other objects, features and advantages of the invention will be made more apparent by considering the following detailed description of an embodiment of the invention and its applications. In the course of this description, frequent reference will be made to the attached drawings.

### BRIEF DESCRIPTION OF THE DRAWINGS

FIG. 1 is a flow chart and schematic illustration of the method of performing protein purification using affinity beads which are held in flow-through perforated wells as contemplated by the invention.

FIG. 2 is a perspective view of a 96-well plate constructed in accordance with the invention;

FIG. 3 is a perspective view of a 96 vessel plate for holding liquids into which the column plate of FIG. 2 is inserted; and

FIG. 4 is a cross-sectional view showing a well with a permeable bottom holding affinity beads inserted into liquid holding vessel.

### DETAILED DESCRIPTION

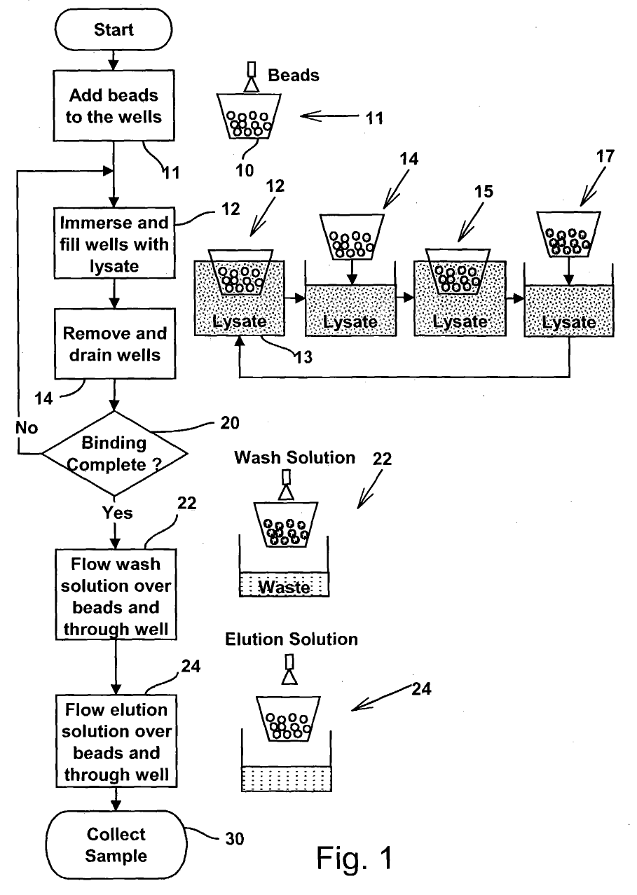

An illustrative embodiment of the invention used to perform high throughput protein purification by affinity methods is shown in FIG. 1.

As contemplated by the invention, one or more wells or cavities having liquid permeable side walls are first filled with affinity beads, such as beads coated with Ni++ for binding proteins tagged with 6× Histidine; beads coated with Glutathione (GLT) for proteins tagged with Glutathione-S-transferase (GST); or beads coated with avidin (monomeric and therefore reversible) for purifying proteins tagged with Biotin; or Immunoprecipitation (beads with either protein A or G) to which an antibody is immobilized to enable binding a protein of interest.

After the wells are filled as indicated generally at 11, they are immersed in lysate solution as indicated at 12 and then removed as seen at 14. When immersed, the lysate flows inwardly into the well through the liquid permeable side walls and/or permeable bottom of the well. When the well is then removed at 14, the lysate is allowed to drain outwardly through the side walls and/or bottom of the well. If the binding of the protein to the affinity beads is not complete, the process is repeated by again immersing the well and the beads in the lysate as seen at 15, and again removing the well from the lysate and allowing it to drain as seen at 17.

The process of immersing and then draining the well is repeated until the binding is complete, as determined by the test seen at 20. A wash solution is then allowed to flow over the beads and through the sidewalls and/or bottom of the well as seen at 22. Alternatively, the plate can be repeatedly dunked in a wash basin.

After the beads are washed, an elution solution is flowed over the beads and through the permeable sidewalls/bottom of the well as seen at 24 before the purified sample is collected as seen at 30. The elution step can also be performed to collect sample fractions.

The total up/down time allotted for binding is determined by the protocol for protein purification using the chosen beads with the well being immersed again each time as soon as it is drained. The act of immersing the well serves to mix the contents of the well (the lysate in the wells and the beads) to promote better binding.

The well which holds the beads is formed such that all or a substantial portion of the bottom of the well is permeable by the liquids that are brought into contact with the beads, including the lysate, the wash solution and the elution buffer. The well may, for example, be formed into a cylindrical or frustoconical cavity with a bottom formed by a 30 micrometer mesh which permits the passage of liquids but not the beads which have a diameter in excess of 30 micrometers. Although the sidewalls of the well may be permeable in addition to or instead of employing a permeable bottom, it has been found that making the bottom permeable and using non-permeable sidewalls provides better mixing and washing than is achieved with permeable sidewalls. The well should be constructed of materials which are inert and hence do not effect the materials being handled. Polypropylene may be used for the sidewalls of the wells and the bottom may be a polyester mesh.

The mesh opening should be as large as possible subject to the constraint that it must contain the beads. The prototype devices proved workable with 25, 33 and 41 micron openings in a woven mesh so these were the real sizes. Note that the beads are not of uniform size and the mesh opening should to be at the low end of the range of possible bead diameters to contain substantially all of the beads. Filter materials other than woven mesh may be used provided the openings are large enough to permit flow by gravity yet sufficiently constricted to retain the small objects being processed

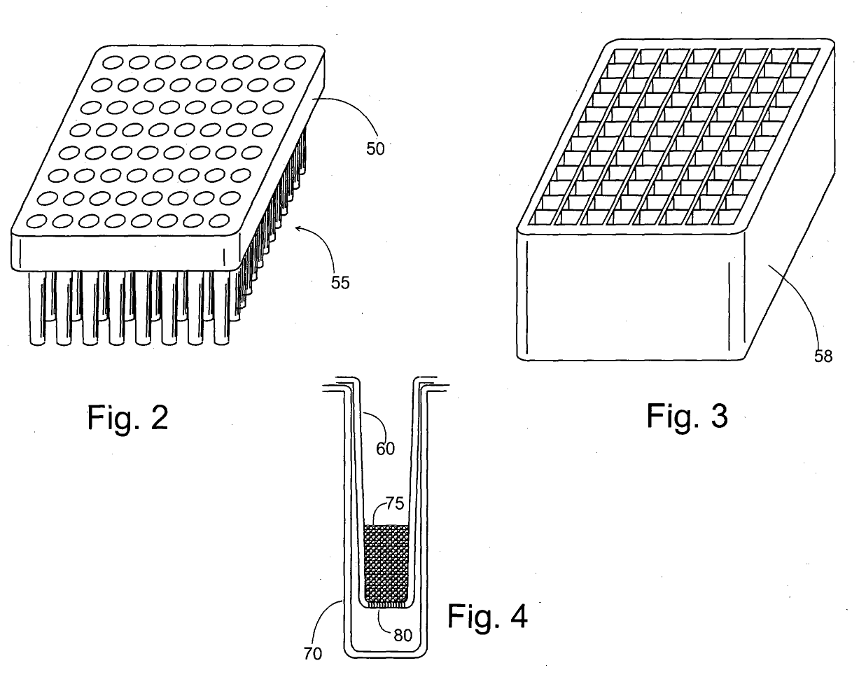

Purification of protein samples is a fundamental need in the field of proteomics and is a basic requirement in a wide variety of academic, clinical, and industrial programs. With the recent advancements in the field of genomics there is an increasing need to express and purify large number of the proteins at the same time. Traditional methods of protein purification are not well suited for high throughput applications. The embodiment of the invention shown in FIGS. 2-4 facilitates the rapid purification of proteins in a high throughput format.

Preferably, a plurality of such wells are held by a single support member, such as the column plate 50 seen in FIG. 2 to which 96 wells indicated generally at 55 are attached. All of the wells 55 attached to the column plate 50 are filled with beads and all are immersed, drained, washed and eluted at the same time to provide a 96-fold increase in the throughput of the system. Alternatively, a plurality of wells arranged in one or more rows may be affixed to a handle and manually, and a plurality of wells may be used in conjunction with a custom workstation or in an automated liquid handling robotic system. In an automated system, the number of times the wells 55 are inserted into vessel containing a liquid, as well as the duration during which the wells are immersed and the duration during which they are allowed to drain, can be precisely controlled to achieve the desired degree of binding. The dipping of the well into a wash or elution buffer during the washing and elution steps can also be timed under program control or, in the alternative, wash or elution buffers may be automatically dispensed in measured amounts into the well from above and allowed to drain through the well into either a plate to catch the elution or waste for wash buffer.

Each of the wells 55 can be inserted into the liquid held in one of the corresponding 96 vessels in a well block 58 shown in FIG. 3. As shown in FIG. 4, each well 60 should be sized and shaped such that it nests into a vessel 70 used to grow the cells that are expressing the proteins. The interior of each vessel in the well block 58 that contains the liquid is larger than, but similar in size to, the exterior of the well holding the small objects so that, when each well is inserted into the corresponding vessel, the level of said liquid in the vessel is displaced to a substantially higher level to increase the liquid pressure at the bottom of said well, increasing the flow rate of the liquid as it is forced upwardly through the permeable bottom of the well. Each well 60 is partially filled with affinity beads as shown at 75. A 40 uM polyethylene mesh 80 attached to the bottom of each well 60 and supports the beads 75 while allowing liquid in the vessel 70 to flow into and out of the well 60 as the well 60 is repeatedly inserted into and withdrawn from the vessel 70. The mesh 80 allows free flow of sample and buffers into and out of the column of while retaining the affinity media. The column plate nests into a standard 96 well deep well plate.

This platform can be utilized to capture proteins by affinity resins or to deplete samples thereby concentrating the specific protein of interest. The embodiment of the invention shown in FIGS. 2-4 was tested to purify His tagged Green fluorescent protein expressed in E. coli using the TALON Cell Thru resin. The cells can be grown, pelleted and lysed in the same well block. The affinity purification using the TALON beads in the column plate was straightforward and the whole process of binding, wash and elution could be performed in less than 20 minutes. There was no requirement for filtration plates or vacuum manifolds.

As an example, E. coli (BL21-SI, Invitrogen) alone or expressing pGFPuv (available from Becton, Dickinson and Company, BD Biosciences Clontech) were grown on LB agar w/Ampicillin selection (100 μg/ml) at 37° C. Colony picked and added to 800 μL of LB liquid media in each well of a 96 well deep well block. IPTG was added to 1 mM to induce expression of GFP. The OD600 is adjusted to 1.5 and the block is spun to pellet the cells. TALON immobilized metal affinity resin (also from available from BD Biosciences Clontech) was used to purify 6x-His tagged GFP. 50 μL of TALON was equilibrated w/lysis buffer and added to each well of the Column plate (slide 8).

Three additional deep well blocks were prepared for the wash step w/400 μL of wash buffer in each well. One deep well block for elution was prepared w/400 μL of the elution buffer in each well.

In the extraction of protein, bacterial pellets were resuspended in the lysis buffer and kept at room temperature for 15 minutes. The lysate was centrifuged (in the plate) at 10,000 rpm for 5 minutes.

In the binding step, the 96 well column plate containing the TALON resin was repeatedly dipped in the supernatant samples and then transferred to the plates containing the wash buffer. Each dipping cycle consumed 32 seconds: 30 seconds in the lysate and 2 seconds out of lysate allowing the wells to drain.

Washing was done by dipping the plate 6 times in a 96 well block containing 400 μl of wash buffer. The dipping cycle was 32 seconds: 30 seconds in the wash buffer and 2 seconds out of the wash buffer. This step was repeated in the three plates containing the wash buffer.

In the elution step, the column plate was transferred to the well block containing the elution buffer and dunked for 4 minutes (the elution dipping cycle was 32 seconds: 30 seconds in the elution buffer and 2 seconds out of the elution buffer.

The samples from the different purification steps were analyzed by SDS PAGE.

When the insert plate was dipped in the sample plate for various residence times, the results suggested that the optimal binding can be achieved within 4 to 6 minutes.

To determine the effect of sample dilution, bacterial cell lysate was diluted with extraction buffer to various volumes in the deep well plate. The total protein amount was kept the same in each well. The results suggest that purification is dependent on the sample concentration. The purity of the eluted sample improves as the dilution of the cell lysate increases.

The methods and apparatus contemplated by the invention allow the user to rapidly purify proteins in a high throughput format on small samples in a short interval of time without the need of elaborate filtration steps or use of vacuum manifolds and the method is compatible with existing automation equipment.

In an automated system, pressure and/or centrifugal force may be applied to speed flow rates and the wells may be agitated to improve mixing. However, it has been found that excellent performance may be achieved without increasing the cost of and complexity of the system by employing such techniques. Employing a pressure assisted device when the invention is employed in a custom manual pipetting device should not, however, have any significant cost impact and it could be expected to improve the performance of the device.

The principles of the invention may also be used in the processing of other kinds of small objects intermixed with other kinds of liquids. For example, small organisms, such as embryos, larvae, or the like that are being stained or otherwise washed/treated with a pharmaceutical, or being exposed to a set of biochemical conditions (e.g. liquids with varying pH, temperature, concentration of salt or similar conditions).

The invention may also be used for immunoprecipitation (IP). In an IP, affinity tagging is accomplished by attaching an antibody to protein A or protein G which have been immobilized on the beads placed in the well. The beads in the permeable wells are intermixed as described above with a liquid containing an immunoprecipitating antibody. The optimal amount of antibody that will quantitatively immunoprecipitate the protein of interest, as well as the mixing time and the incubation time, should be empirically determined for each cell model. Using the invention to perform immunoprecipitation provides an efficient analytical method for pulling out the protein of interest, and anything else that may be bound to the protein of interest.

### CONCLUSION

It should be understood that the methods and apparatus which have been described are merely illustrative applications of the principles of the invention. Numerous modifications may be made to the arrangements described without departing from the true spirit and scope of the invention.

1. Apparatus for intermixing small objects and a liquid comprising at least one receptacle for receiving and retaining said small objects, at least a portion of said receptacle being permeable to permit said liquid to flow through said portion to intermix with said small objects when said receptacle is inserted into a vessel containing said liquid.

2. Apparatus for intermixing small objects and a liquid as set forth in claim 1 wherein said small objects are affinity beads and said liquid is a lysate containing proteins.

3. Apparatus for intermixing small objects and a liquid as set forth in claim 1 further including means for immersing said receptacle in said vessel a plurality of times.

4. Apparatus for intermixing small objects and a liquid as set forth in claim 1 wherein said receptacle is constructed of a material that is chemically inert with respect to said liquid and said small objects.

5. Apparatus for intermixing small objects and a liquid as set forth in claim 1 wherein said portion of said receptacle that is permeable is located at or near the bottom of said receptacle.

6. Apparatus for intermixing small objects and a liquid as set forth in claim 1 wherein said portion of said receptacle that is permeable is formed from a mesh having openings that are smaller than substantially all of said small objects to permit the flow of said liquid therethrough while retaining said small objects within said receptacle.

7. Apparatus for intermixing small objects and a liquid as set forth in claim 1 wherein said portion of said receptacle that is permeable comprises a substantial portion of the bottom or lower the lower sidewalls of said receptacle.

8. Apparatus for intermixing small objects and a liquid as set forth in claim 1 wherein said small objects comprise one or more living organisms.

9. Apparatus for intermixing small objects and a liquid as set forth in claim 8 wherein said liquid contains a stain or a pharmaceutical, or said living organisms are subjected to biochemical conditions, whose effect on said living organism is being studied.

10. Apparatus for intermixing small objects and a liquid as set forth in claim 1 wherein the interior of said vessel containing said liquid is larger than but similar in size to the exterior of said receptacle so that, when said receptacle is inserted into said vessel, the level of said liquid in said vessel is displaced to a substantially higher level to increase the liquid pressure at the bottom of said receptacle to force said liquid upwardly into said permeable portion of said receptacle.

11. Apparatus for intermixing small objects and a liquid as set forth in claim 1 wherein said small objects carry proteins and wherein said liquid contains an antibody which attaches to said proteins to cause immunoprecipitation.

12. Apparatus for intermixing small objects with a liquid comprising
a plurality of receptacles containing said small objects, each of said receptacles having a lower portion thereof that is permeable to permit the flow of said liquid therethrough,
a vessel containing said solution, and
means for repeatedly inserting said receptacles into said liquid in said vessel and then withdrawing said receptacle from said liquid to cause said liquid to first flow into said receptacle through said lower portion when said receptacle is inserted into said liquid and then to flow outwardly from said receptacle through said lower portion when said receptacle is withdrawn from said liquid.

13. Apparatus for intermixing small objects with a liquid as set forth in claim 12 wherein said liquid is a lysate containing proteins and said small objects are affinity beads to which said proteins become bound when said liquid and said affinity beads are intermixed.

14. Apparatus for intermixing small objects with a liquid as set forth in claim 13 wherein said portion of each of said receptacles that is permeable defines openings which are large enough to permit the flow of said lysate therethrough but smaller than substantially all of said affinity beads.

15. Apparatus for intermixing small objects with a liquid as set forth in claim 12 wherein said portion of each of said receptacles that is permeable defines openings which are large enough to permit the flow of said liquid therethrough but smaller than substantially all of said solid objects.

16. Apparatus for intermixing small objects with a liquid as set forth in claim 15 wherein said portion of said receptacle that is permeable is a mesh located at the bottom of said receptacle.

17. Apparatus for intermixing small objects with a liquid as set forth in claim 12 wherein said small objects are affinity beads and said liquid is a lysate containing a protein to purified.

18. Apparatus for intermixing small objects with a liquid as set forth in claim 12 wherein said small objects are living organisms and said liquid contains a stain or pharmaceutical whose effect on said living organisms is being studied.
